(19) United States
(12) Patent Application Publication
(10) Pub. No.: US 2021/0220189 A1
(43) Pub. Date: Jul. 22, 2021

(54) ABSORBENT ARTICLE WITH BODY CONFORMING STRUCTURE

(71) Applicant: Kimberly-Clark Worldwide, Inc., Neenah, WI (US)

(72) Inventors: Lin Miao, Beijing (CN); Xiaoling Huang, Shanghai (CN); Lilian Ma, Shanghai (CN); Siyan Liu, Shanghai (CN); Tingting Wan, Shanghai (CN); Yi Chen, Shanghai (CN); Beibei Su, Shanghai (CN)

(21) Appl. No.: 17/056,215

(22) PCT Filed: May 29, 2018
(86) PCT No.: PCT/CN2018/088790

§ 371 Date: Nov. 17, 2020

(51) Int. Cl. A61F 13/534; A61F 13/472; A61F 13/47

(52) U.S. Cl. CPC A61F 13/53409; A61F 2013/530883; A61F 13/4704; A61F 13/47218

(57) ABSTRACT

An absorbent article (10) is disclosed. The absorbent article (10) can have a topsheet layer (30), a liquid impermeable layer (36), and an absorbent assembly (40) positioned between the topsheet layer (30) and the liquid impermeable layer (36). The absorbent assembly (40) can have a body conforming layer (38) which has a plurality of slits (50) and at least one crease (60) extending in the longitudinal direction (X). The presence of the plurality of slits (50) and the crease (60) in the body conforming layer (38) can provide for a reduction in the stiffness of the absorbent assembly (40). The absorbent article (10) can deform under pressure and have an improvement in conformity to the body of the wearer of the absorbent article (10).

### BACKGROUND OF THE DISCLOSURE

Products such as absorbent articles are often used to collect and retain human body exudates containing, for example, urine, menses, and/or blood. Comfort, absorbency, and discretion are three main product attributes and areas of concern for the wearer of the absorbent article. In particular, a wearer is often interested in knowing that such products will absorb significant volumes of body exudates with minimal leakage in order to protect their undergarments, outer garments, or bedsheets from staining, and that such products will help them avoid the subsequent embarrassment brought on by such staining.

Currently, a wide variety of products for absorption of body exudates are available in the form of diapers, training pants, feminine pads, sanitary napkins, panty shields, pantiliners, and incontinence devices. These products generally have an absorbent core positioned between a body-facing liquid permeable topsheet layer and a garment-facing liquid impermeable layer. The edges of the topsheet layer and the liquid impermeable layer are often bonded together at their periphery to form a seal to contain the absorbent core and body exudates received into the product through the topsheet layer. In use, products such as, for example, feminine pads and sanitary napkins are typically positioned in the crotch portion of an undergarment for absorption of the body exudates and a garment attachment adhesive on the liquid impermeable layer can be used to attach the product to the inner crotch portion of the undergarment. Some of these products can also include wing-like structures for wrapping about the wearer's undergarment to further secure the product to the undergarment and to protect the undergarment from staining. Such wing-like structures (also known as flaps or tabs) are frequently made from lateral extensions of the topsheet and/or liquid impermeable layers.

One problem with such conventional absorbent articles is that the absorbent articles may not always have an adequate fit to the body of the wearer which can lead to increased levels of leakage of body exudates from the absorbent article and discomfort during wear of the absorbent article. Many conventional absorbent articles are flat or have flat regions prior to use while the wearers body is contoured. Even though the flat absorbent article can bend during use, they can fail to fully conform to the body of the wearer which can result in gaps between the absorbent article and the skin of the wearer resulting in leakage of body exudates. The movement of the wearer can also cause undesirable deformation, such as, for example, bunching, twisting and/or roping, of the absorbent article and fold lines within the absorbent article which can create pathways along which the body exudates can travel and leak from the absorbent article.

As a result, there remains a need for an improved product, such as an absorbent article, that has an improved conformance to the body of the wearer.

### SUMMARY OF THE DISCLOSURE

In various embodiments, an absorbent article can have a topsheet layer; a liquid impermeable layer; an absorbent assembly positioned between the topsheet layer and the liquid impermeable layer and comprising a body conforming layer wherein the body conforming layer comprises a crease and a first plurality of slits. In various embodiments, the first plurality of slits are arranged randomly in the body conforming layer. In various embodiments, the first plurality of slits are arranged in a longitudinal direction of the absorbent article. In various embodiments, the first plurality of slits are arranged in a first longitudinal direction column. In various embodiments, the body conforming layer further comprises a second plurality of slits arranged in a longitudinal direction of the absorbent article. In various embodiments, the second plurality of slits are arranged in a second longitudinal direction column. In various embodiments, the first plurality of slits arranged in the first longitudinal direction column are offset in a transverse direction of the absorbent article from the second plurality of slits arranged in the second longitudinal direction column. In various embodiments, the first plurality of slits arranged in the first longitudinal direction column at least partially overlap in a transverse direction of the absorbent article the second plurality of slits arranged in the second longitudinal direction column.

In various embodiments, the crease is centered on a longitudinal direction centerline of the absorbent article. In various embodiments, the first plurality of slits overlap the crease.

In various embodiments, the absorbent assembly further comprises a second absorbent layer positioned between the topsheet layer and the body conforming layer. In various embodiments, the absorbent assembly further comprises a second absorbent layer positioned between the body conforming layer and the liquid impermeable layer. In various embodiments, the absorbent article further has a fluid intake layer positioned between the topsheet layer and the absorbent assembly. In various embodiments, the absorbent article further has a fluid distribution layer positioned between the topsheet layer and the absorbent assembly.

In various embodiments, the absorbent article further has a first flexure in a posterior region of the absorbent article extending in a direction parallel to a longitudinal centerline. In various embodiments, the absorbent article further has a second flexure in the posterior region of the absorbent article and spaced transverse outward in a first direction from the first flexure, the second flexure defining a first side portion of the absorbent article transversely outward in the first direction from the second flexure, and a third flexure in the posterior region of the absorbent article and spaced transversely outward in a second direction from the first flexure, the second direction being opposite the first direction, the third flexure defining a second side portion of the absorbent article transversely outward in the second direction from the third flexure, the second and third flexures defining a central portion of the absorbent article therebetween.

### BRIEF DESCRIPTION OF DRAWINGS

FIG. 1 is a top plan view of an embodiment of an absorbent article.

FIG. 2 is a top plan view of the absorbent assembly of the absorbent article of FIG. 1.

FIG. 3 is an end view of the absorbent assembly of the absorbent article of FIG. 1.

FIG. 4 is an exploded perspective view of the absorbent article of FIG. 1.

FIG. 5 is a top plan view of another embodiment of an absorbent article.

FIG. 6 is a top plan view of the absorbent article of FIG. 5 with the topsheet layer removed to illustrate underlying structure.

FIG. 7 is a top plan view of the absorbent assembly of the absorbent article of FIG. 5.

FIG. 8 is a bottom plan view of the absorbent assembly of the absorbent article of FIG. 5.

FIG. 9 is an exploded end view of the absorbent assembly of FIG. 7

FIG. 10 is a top plan view of another embodiment of an absorbent article.

FIG. 11 is an exploded perspective view of the absorbent article of FIG. 10.

FIG. 12 is an exploded cross-sectional view of the absorbent article of FIG. 10 taken along line 12-12.

FIG. 13 is a bottom plan view of an embodiment of an absorbent article.

FIG. 14 is a bottom plan view of another embodiment of an absorbent article.

FIG. 15 is a magnified view of the area of FIG. 10 bound by the dot-dash rectangle A.

FIG. 16 is a perspective view of the absorbent article of FIG. 10 in a tented configuration.

Repeat use of reference characters in the present specification and drawings is intended to represent the same or analogous features or elements of the disclosure.

### DETAILED DESCRIPTION OF THE DISCLOSURE

The present disclosure is directed towards an absorbent article which can have an improved conformity to the body of the wearer of the absorbent article. An absorbent article can have a longitudinal direction, a transverse direction, and a depth direction. The absorbent article can have an anterior region, a posterior region, and a central region. The absorbent article can have a topsheet layer, a liquid impermeable layer, and an absorbent assembly positioned between the topsheet layer and the liquid impermeable layer. In various embodiments, the absorbent assembly can have a body conforming layer wherein the body conforming layer has a plurality of slits extending from a body facing surface of the body conforming layer to the garment facing surface of the body conforming layer and at least one crease extending in the longitudinal direction of the body conforming layer. The presence of the plurality of slits and the crease in the body conforming layer of the absorbent assembly can provide for a reduction in the stiffness of the absorbent assembly therefore allowing the absorbent article to deform under pressure and have an improvement in conformity to the body of the wearer of the absorbent article. The presence of the slits and the crease in the body conforming layer can allow the absorbent article to take and maintain a tented configuration throughout the length of the absorbent article when the wearer moves, such as walking, running, exercising, etc. The tented configuration conforms the absorbent article to the body of the wearer and the absorbent article moves in response to the alternating movement of the legs of the wearer. The tented configuration of the absorbent article can allow the absorbent article to maintain intimate contact with the body of the wearer while minimizing bunching, twisting, and roping.

### Definitions

As used herein, the term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, training pants, youth pants, swim pants, feminine hygiene products including, but not limited to, menstrual pads, sanitary napkins, feminine pads, pantiliners, and panty shields, and incontinence products, and the like without departing from the scope of the present disclosure.

As used herein, the term “airlaid” refers herein to a web manufactured by an airlaying process In the airlaying process, bundles of small fibers having typical lengths ranging from about 3 to about 52 mm are separated and entrained in an air supply and then deposited onto a forming screen, usually with the assistance of a vacuum supply. The randomly deposited fibers are then bonded to one another using, for example, hot air to activate a binder component or a latex adhesive. Airlaying is taught in, for example, U.S. Pat. No. 4,640,810 to Laursen, et al., which is incorporated herein in its entirety by reference thereto for all purposes.

As used herein, the term “bonded” refers to the joining, adhering, connecting, attaching, or the like, of two elements. Two elements will be considered bonded together when they are joined, adhered, connected, attached, or the like, directly to one another or indirectly to one another, such as when bonded to an intermediate element. The bonding can occur via, for example, adhesive, pressure bonding, thermal bonding, ultrasonic bonding, stitching, suturing, and/or welding.

As used herein, the term “bonded carded web” refers herein to webs that are made from staple fibers which are sent through a combing or carding unit which separates or breaks apart and aligns the staple fibers in the machine direction to form a generally machine direction oriented fibrous nonwoven web. This material may be bonded together by methods that can include point bonding, through air bonding, ultrasonic bonding, adhesive bonding, etc.

As used herein, the term “coform” refers herein to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff, and also superabsorbent particles, inorganic and/or organic absorbent materials, treated polymeric staple fibers and so forth. Some examples of such coform materials are disclosed in U.S. Pat. No. 4,100,324 to Anderson, et al., U.S. Pat. No. 4,818,464 to Lau, U.S. Pat. No. 5,284,703 to Everhart, et al., and U.S. Pat. No. 5,350,624 to Georger, et al., each of which are incorporated herein in their entirety by reference thereto for all purposes.

As used herein, the term “conjugate fibers” refers herein to fibers which have been formed from at least two polymer sources extruded from separate extruders and spun together to form on fiber. Conjugate fibers are also sometimes referred to as bicomponent or multicomponent fibers. The polymers are arranged in substantially constantly positioned distinct zones across the cross-sections of the conjugate fibers and extend continuously along the length of the conjugate fibers. The configuration of such a conjugate fiber may be, for example, a sheath/core arrangement where one polymer is surrounded by another, or may be a side-by-side arrangement, a pie arrangement, or an “islands-in-the-sea” arrangement. Conjugate fibers are taught by U.S. Pat. No. 5,108,820 to Kaneko, et al., U.S. Pat. No. 4,795,668 to Krueger, et al., U.S. Pat. No. 5,540,992 to Marcher, et al., U.S. Pat. No. 5,336,552 to Strack, et al., U.S. Pat. No. 5,425,987 to Shawver, and U.S. Pat. No. 5,382,400 to Pike, et al., each being incorporated herein in their entirety by reference thereto for all purposes. For two component fibers, the polymers may be present in ratios of 75/25, 50/50, 25/75 or any other desired ratio. Additionally, polymer additives such as processing aids may be included in each zone.

As used herein, the term “machine direction” (MD) refers to the length of a fabric in the direction in which it is produced, as opposed to a “cross-machine direction” (CD) which refers to the width of a fabric in a direction generally perpendicular to the machine direction.

As used herein, the term “meltblown web” refers herein to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Buten, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “nonwoven fabric” or “nonwoven web” refers herein to a web having a structure of individual fibers or threads which are interlaid, but not in an identifiable manner as in a knitted fabric. Nonwoven fabrics or webs have been formed from many processes such as, for example, meltblowing processes, spunbonding processes, through-air bonded carded web (also known as BCW and TABCW) processes, etc. The basis weight of nonwoven webs may generally vary, such as, from about 5, 10, or 20 gsm to about 120, 125, or 150 gsm.

As used herein, the term “spunbond web” refers herein to a web containing small diameter substantially continuous fibers. The fibers are formed by extruding a molten thermoplastic material from a plurality of fine, usually circular, capillaries of a spinneret with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are each incorporated herein in their entirety by reference thereto for all purposes. Spunbond fibers are generally not tacky when they are deposited onto a collecting surface. Spunbond fibers may sometimes have diameters less than about 40 microns, and often between about 5 to about 20 microns.

As used herein, the terms “superabsorbent polymer,” “superabsorbent,” or “SAP” shall be used interchangeably and shall refer to polymers that can absorb and retain extremely large amounts of a liquid relative to their own mass. Water absorbing polymers, which are classified as hydrogels, which can be cross-linked, absorb aqueous solutions through hydrogen bonding and other polar forces with water molecules. A SAP's ability to absorb water is based in par on iconicity (a factor of the ionic concentration of the aqueous solution), and the SAP functional polar groups that have an affinity for water. SAP are typically made from the polymerization of acrylic acid blended with sodium hydroxide I the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. SAP may be present in absorbent articles in particle or fibrous form or as a coating or another material or fiber.

Absorbent Article:

The present disclosure is directed towards an absorbent article which can have an improved conformity to the body of the wearer of the absorbent article. An absorbent article can have a longitudinal direction, a transverse direction, and a depth direction. The absorbent article can have an anterior region, a posterior region, and a central region. The absorbent article can have a topsheet layer, a liquid impermeable layer, and an absorbent assembly positioned between the topsheet layer and the liquid impermeable layer. In various embodiments, the absorbent assembly can have a body conforming layer wherein the body conforming layer has a plurality of slits extending from a body facing surface of the body conforming layer to a garment facing surface of the body conforming layer and at least one crease extending in the longitudinal direction of the body conforming layer. The presence of the plurality of slits and the crease in the body conforming layer of the absorbent assembly can provide for a reduction in the stiffness of the absorbent assembly therefore allowing the absorbent article to deform under pressure and have an improvement in conformity to the body of the wearer of the absorbent article. The presence of the slits and the crease in the body conforming layer can allow the absorbent article to take and maintain a tented configuration throughout the length of the absorbent article when the wearer moves, such as when walking, running, exercising, etc. The tented configuration conforms the absorbent article to the body of the wearer and the absorbent article moves in response to the alternating movement of the legs of the wearer. The tented configuration of the absorbent article can allow the absorbent article to maintain intimate contact with the body of the wearer while minimizing bunching, twisting, and roping.

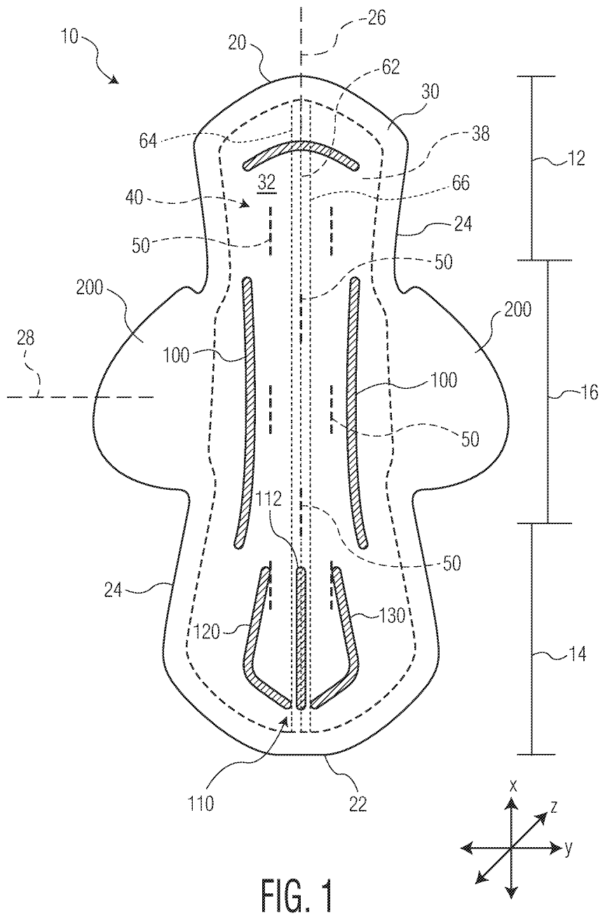

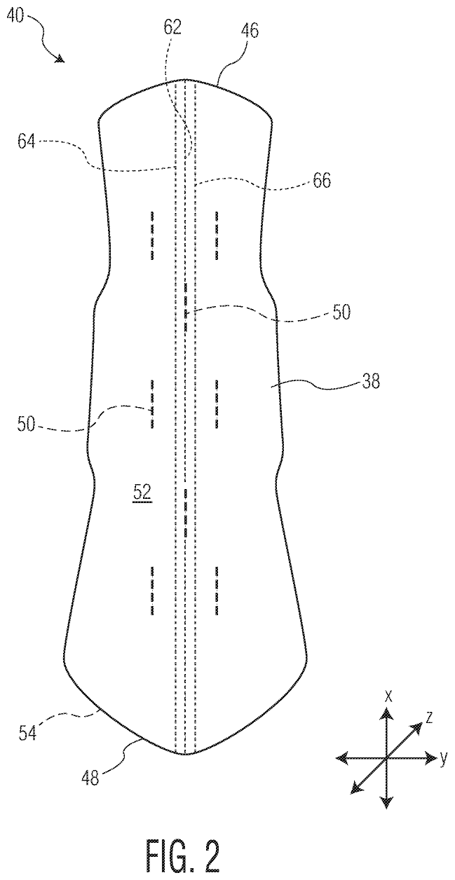

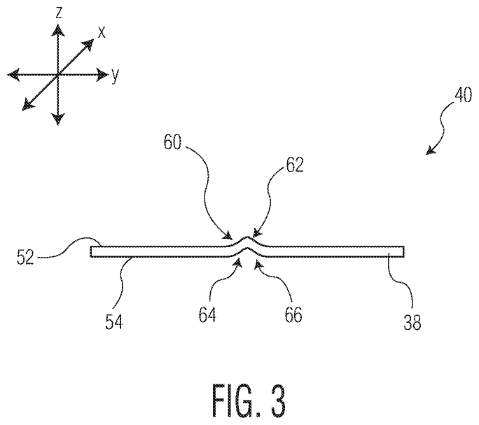

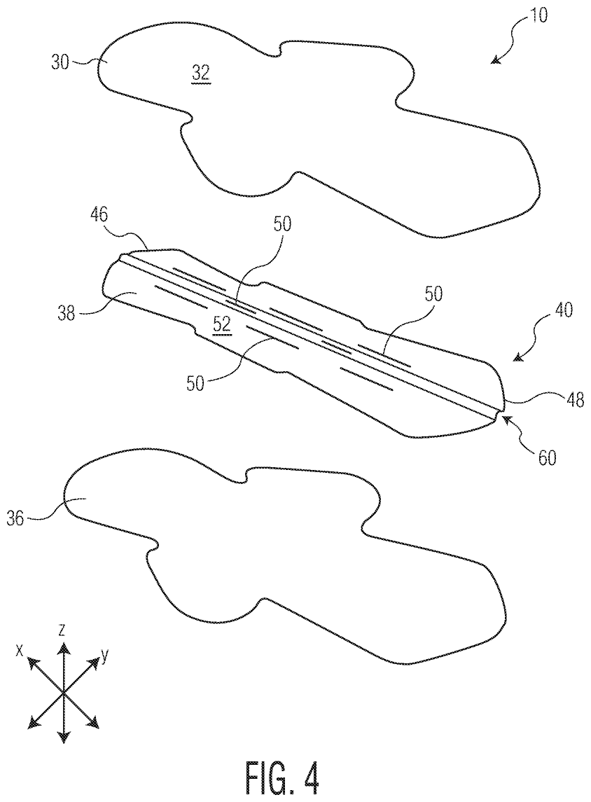

Referring to FIGS. 1-4, an exemplary embodiment of an absorbent article 10 is illustrated in various views and configurations. FIG. 1 provides a top plan view of an illustration of an embodiment of an absorbent article 10 of the present disclosure in the form of a feminine hygiene product such as a menstrual pad or sanitary napkin. It is to be understood that the present disclosure is suitable for use with various other absorbent articles, such as, but not limited to, diapers or incontinence products, without departing from the scope of the present disclosure. FIG. 2 provides a top plan view of the absorbent assembly 40 of the absorbent article 10 of FIG. 1. FIG. 3 provides an end view of the absorbent assembly 40 of FIG. 2. FIG. 4 provides an exploded perspective view of the absorbent article 10 of FIG. 1. The absorbent article 10 can have a longitudinal direction (X), a transverse direction (Y), and a depth direction (Z). The absorbent article 10 can have an anterior region 12, a posterior region 14, and a central region 16 located between the anterior region 12 and the posterior region 14. In general, the anterior region 12 is adapted to be worn towards the front of the wearer, the posterior region 14 is adapted to be worn toward the rear of the wearer, and the central region 16 is adapted to be worn proximate to the wearer's crotch. The absorbent article 10 can have a first transverse direction end edge 20, a second transverse direction end edge 22 opposed to the first transverse direction end edge 20, and a pair of opposing longitudinal direction side edges 24 extending between and connecting the first and second transverse direction end edges, 20 and 22. The absorbent article 10 can have a longitudinal centerline 26 and a transverse centerline 28. The absorbent article 10 can have a wearer facing, liquid permeable topsheet layer 30 and a garment facing, liquid impermeable layer 36. An absorbent assembly 40 can be positioned between the topsheet layer 30 and the liquid impermeable layer 36. The topsheet layer 30 and the liquid impermeable layer 36 can both extend beyond the outermost peripheral edges of the absorbent assembly 40 and can be peripherally bonded together, either entirely or partially, using known bonding techniques to form a sealed peripheral region. For example, the topsheet layer 30 and the liquid impermeable layer 36 can be bonded together by adhesive bonding, ultrasonic bonding, or any other suitable bonding technique known in the art.

Each of these components of the absorbent article 10, as well as additional components, will be described in more detail herein.

Topsheet Layer:

The topsheet layer 30 defines a body facing surface 32 of the absorbent article 10 that may directly contact the body of the wearer and is liquid permeable to receive body exudates. The topsheet layer 30 is desirably provided for comfort and functions to direct body exudates away from the body of the wearer, through its own structure, and towards the absorbent assembly 40. The topsheet layer 30 desirably retains little to no liquid in its structure, so that it provides a relatively comfortable and non-irritating surface next to the skin of the wearer of the absorbent article 10.

The topsheet layer 30 can be a single layer of material, or alternatively, can be multiple layers that have been laminated together. The topsheet layer 30 can be constructed of any material such as one or more woven sheets, one or more fibrous nonwoven sheets, one or more film sheets, such as blown or extruded films, which may themselves be of single or multiple layers, one or more foam sheets, such as reticulated, open cell or closed cell foams, a coated nonwoven sheet, or a combination of any of these materials. Such combination can be adhesively, thermally, or ultrasonically laminated into a unified planar sheet structure to form a topsheet layer 30.

In various embodiments the topsheet layer 30 can be constructed from various nonwoven webs such as meltblown webs, spunbond webs, hydroentangled spunlace webs, or through air bonded carded webs. Examples of suitable topsheet layer 30 materials can include, but are not limited to, natural fiber webs (such as cotton), rayon, hydroentangled webs, bonded carded webs of polyester, polypropylene, polyethylene, nylon, or other heat-bondable fibers (such as bicomponent fibers), polyolefins, copolymers of polypropylene and polyethylene, linear low-density polyethylene, and aliphatic esters such as polylactic acid. Finely perforated films and net materials can also be used, as can laminates of/or combinations of these materials. An example of a suitable topsheet layer 30 can be a bonded carded web made of polypropylene and polyethylene such as that obtainable from Sandler Corp., Germany. U.S. Pat. No. 4,801,494 to Datta, et al., and U.S. Pat. No. 4,908,026 to Sukiennik, et al., and WO 2009/062998 to Texol teach various other topsheet materials that may be used as the topsheet layer 30, each of which is hereby incorporated by reference thereto in its entirety. Additional topsheet layer 30 materials can include, but are not limited to, those described in U.S. Pat. No. 4,397,644 to Matthews, et al., U.S. Pat. No. 4,629,643 to Curro, et al., U.S. Pat. No. 5,188,625 to Van Iten, et al., U.S. Pat. No. 5,382,400 to Pike, et al., U.S. Pat. No. 5,533,991 to Kirby, et al., U.S. Pat. No. 6,410,823 to Daley, et al., and U.S. Publication No. 2012/0289917 to Abuto, et al., each of which is hereby incorporated by reference thereto in its entirety.

In various embodiments, the topsheet layer 30 may contain a plurality of apertures formed therethrough to permit body exudates to pass more readily into the absorbent assembly 40. The apertures may be randomly or uniformly arranged throughout the topsheet layer 30. The size, shape, diameter, and number of apertures may be varied to suit an absorbent article's 10 particular needs.

In various embodiments, the tospheet layer 30 can have a basis weight ranging from about 5, 10, 15, 20, or 25 gsm to about 50, 100, 120, 125, or 150 gsm. For example, in an embodiment, a topsheet layer 30 can be constructed from a through air bonded carded web having a basis weight ranging from about 15 gsm to about 100 gsm. In another example, a topsheet layer 30 can be constructed from a through air bonded carded web having a basis weight from about 20 gsm to about 50 gsm, such as a through air bonded carded web that is readily available from nonwoven material manufacturers.

In various embodiments, the topsheet layer 30 can be at least partially hydrophilic. In various embodiments, a portion of the topsheet layer 30 can be hydrophilic and a portion of the topsheet layer 30 can be hydrophobic. In various embodiments, the portions of the topsheet layer 30 which can be hydrophobic can be either an inherently hydrophobic material or can be a material treated with a hydrophobic coating.

In various embodiments, the topsheet layer 30 can be a multicomponent topsheet layer 30 such as by having two or more different nonwoven or film materials, with the different materials placed in separate locations in the transverse direction (Y) of the absorbent article 10. For example, the topsheet layer 30 can be a two layer or multicomponent material having a central portion positioned along and straddling a longitudinal centerline 26 of an absorbent article 10, with lateral side portions flanking and bonded to each side edge of the central portion. The central portion can be constructed from a first material and the side portions can be constructed from a material which can be the same as or different from the material of the central portion. In such embodiments, the central portion may be at least partially hydrophilic and the side portions may be inherently hydrophobic or may be treated with a hydrophobic coating. Examples of constructions of multi-component topsheet layers 30 are generally described in U.S. Pat. No. 5,961,505 to Coe, U.S. Pat. No. 5,415,640 to Kirby, and U.S. Pat. No. 6,117,523 to Sugahara, each of which is incorporated herein by reference thereto in its entirety.

In various embodiments, a central portion of a topsheet layer 30 can be positioned symmetrically about the absorbent article 10 longitudinal centerline 26. Such central longitudinally directed central portion can be a through air bonded carded web (“TABCW”) having a basis weight between about 15 and about 100 gsm. Previously described nonwoven, woven, and aperture film topsheet layer materials may also be used as the central portion of a topsheet layer 30. In various embodiments, the central portion can be constructed from a TABCW material having a basis weight from about 20 gsm to about 50 gsm such as is readily available from nonwoven material manufacturers. Alternatively, aperture films, such as those available from such film suppliers as Texol, Italy and Tredegar, U.S.A. may be utilized. Different nonwoven, woven, or film sheet materials may be utilized as the side portions of the topsheet layer 30. The selection of such topsheet layer 30 materials can vary based upon the overall desired attributes of the topsheet layer 30. For example, it may be desired to have a hydrophilic material in the central portion and hydrophobic-barrier type materials in the side portions to prevent leakage and increase a sense of dryness in the area of the side portions. Such side portions can be adhesively, thermally, ultrasonically, or otherwise bonded to the central portion along or adjacent the longitudinally directed side edges of the central portion. Traditional absorbent article construction adhesive may be used to bond the side portions to the central portion. Either of the central portion and/or the side portions may be treated with surfactants and/or skin-health benefit agents, as are well known in the art.

Such longitudinally directed side portions can be of a single or multi-layered construction. In various embodiments, the side portions can be adhesively or otherwise bonded laminates. In various embodiments, the side portions can be constructed of an upper fibrous nonwoven layer, such as a spunbond material, laminated to a bottom layer of a hydrophobic barrier film material. Such a spunbond layer may be formed from a polyolefin, such as a polypropylene and can include a wetting agent if desired. In various embodiments, a spunbond layer can have a basis weight from about 10 or 12 gsm to about 30 or 70 gsm and can be treated with hydrophilic wetting agents. In various embodiments, a film layer may have apertures to allow fluid to permeate to lower layers, and may be either of a single layer or multi-layer construction. In various embodiments, such film can be a polyolefin, such as polyethylene having a basis weight from about 10 to about 40 gsm. Construction adhesive can be utilized to laminate the spunbond layer to the film layer at an add-on level of between about 0.1 gsm and 15 gsm. When a film barrier layer is used in the overall topsheet layer 30 design, it may include opacifying agents, such as film pigments, that can help the film in masking stains along the absorbent article 10 side edges, thereby serving as a masking element. In such a fashion, the film layer can serve to limit visualization of a fluid insult stain along the absorbent article 10 side edges when viewed from above the topsheet layer 30. The film layer may also serve as a barrier layer to prevent rewet of the topsheet layer 30 as well as to prevent the flow of fluid off the side edges of the absorbent article 10. In various embodiments, the side portions can be laminates such as a spunbond-meltblown-meltblown-spunbond layer (“SMMS”) laminate, spunbond-film laminate, or alternatively, other nonwoven laminate combinations.

Absorbent Assembly:

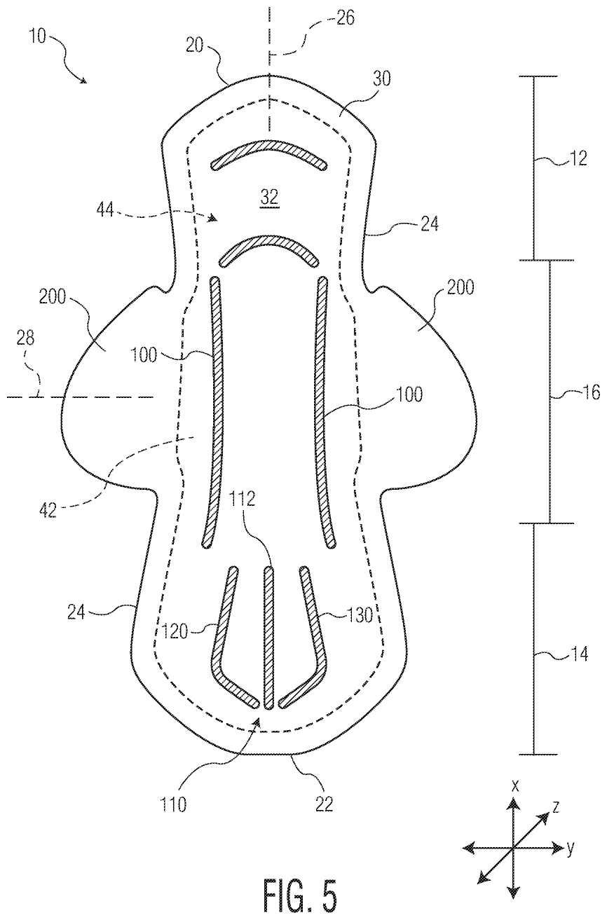

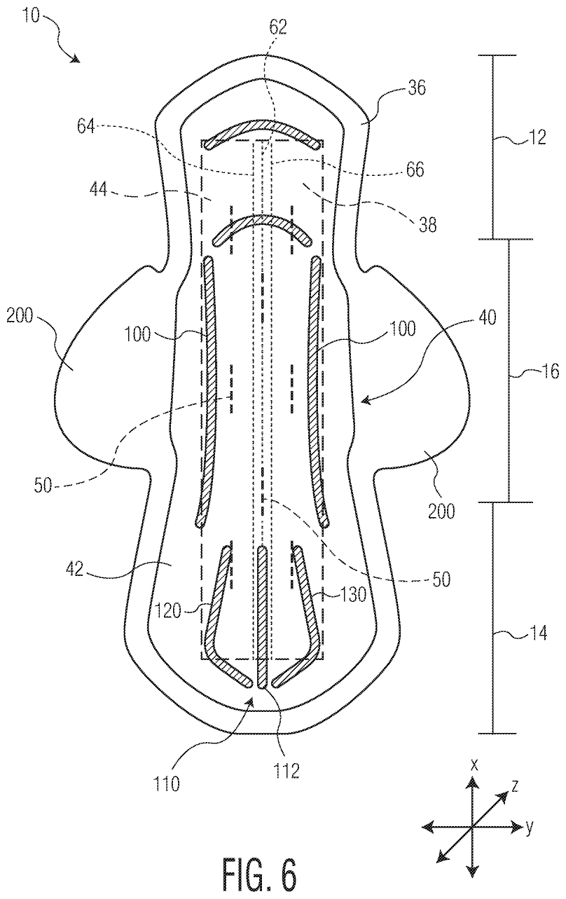

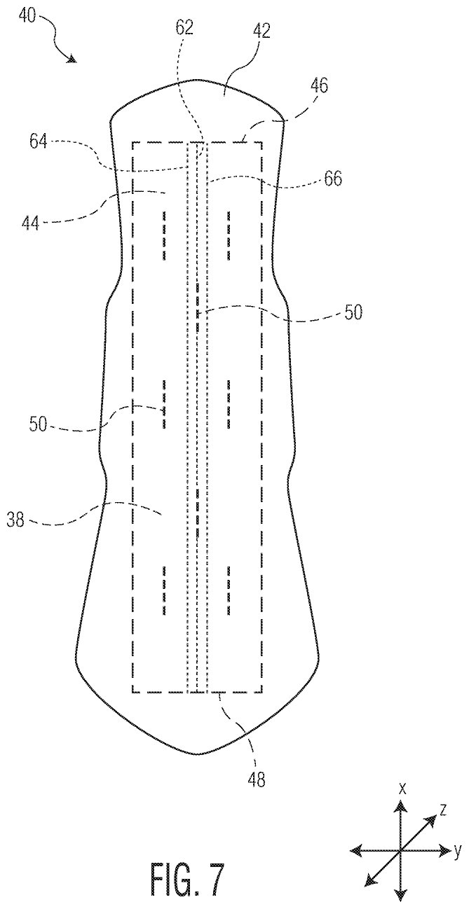

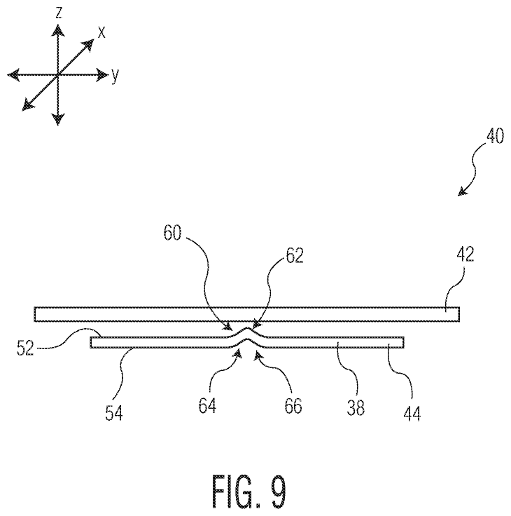

An absorbent assembly 40 can be positioned between the topsheet layer 30 and the liquid impermeable layer 36 of the absorbent article 10. In various embodiments, such as, for example, illustrated in FIGS. 1-4, the absorbent assembly 40 can have a single layer. In various embodiments, such as, for example, illustrated in FIGS. 5-9, the absorbent assembly 40 can have two layers, such as, for example, an upper absorbent layer 42 and a lower absorbent layer 44 in fluid communication with each other. At least one of the absorbent assembly 40 is a body conforming layer 38. In various embodiments in which the absorbent assembly 40 has a single layer, the single layer of the absorbent assembly 40 is the body conforming layer 38 such as, for example, illustrated in FIGS. 1-4. In various embodiments in which the absorbent assembly 40 has an upper absorbent layer 42 and a lower absorbent layer 44, either of the upper absorbent layer 42 or the lower absorbent layer 44 of the absorbent assembly 40 is a body conforming layer 38. In the exemplary embodiment illustrated in FIGS. 5-9, the lower absorbent layer 44 is illustrated as the body conforming layer 38. In various embodiments, the upper absorbent layer 42 can be the body conforming layer 38.

In various embodiments, the body conforming layer 38 can have a high bending modulus. In various embodiments in which the absorbent assembly 40 has an upper absorbent layer 42 and a lower absorbent layer 44, the upper absorbent layer 42 can have a bending modulus at least equal or higher than the lower absorbent layer 44. In such embodiments, the upper absorbent layer 42 can be the body conforming layer 38. In various embodiments in which the absorbent assembly 40 has an upper absorbent layer 42 and a lower absorbent layer 44, the lower absorbent layer 44 can have a have a bending modulus at least equal or higher than the upper absorbent layer 42. In such embodiments, the lower absorbent layer 44 can be the body conforming layer 38.

In various embodiments, the body conforming layer 38 can have a plurality of slits 50. The body conforming layer 38 can have a body facing surface 52 and a garment facing surface 54 and each slit 50 can extend through the body conforming layer 38 from the body facing surface 52 to the garment facing surface 54. Each of the slits 50 is a cut through the thickness of the body conforming layer 38 in the depth direction (Z) of the body conforming layer 38 without removing any of the absorbent materials from the body conforming layer 38. Avoiding the removal of absorbent material during the incorporation of slits 50 can maintain the overall absorbent capacity of the absorbent assembly 40. The slits 50 in the body conforming layer 38 can allow for the material forming the body conforming layer 38 that is adjacent to a slit 50 to shift with the movement of the wearer of the absorbent article 10, thereby providing areas of stress relief to the body conforming layer 38. For example, in various embodiments, a configuration of a body conforming layer 38 can be a superabsorbent polymer-containing compressed sheet which can have a high bending modulus. Providing slits 50 into a superabsorbent polymer-containing compressed sheet having a high bending modulus can provide areas of stress relief to the superabsorbent polymer-containing compressed sheet without removing absorbent material from the superabsorbent polymer-containing compressed sheet. For example, the slits 50 can allow the material forming the body conforming layer 38 that is adjacent to a slit 50 to shift out of plane such that a first portion of the material forming the body conforming layer 38 can overlap a second portion of the material forming the body conforming layer 38. As an additional example, the material forming the body conforming layer 38 that is adjacent to a slit 50 can buckle and deform without necessarily overlapping another portion of the material forming the body conforming layer 38.

Each slit 50 within the body conforming layer 38 can be separate and independent from each other slit 50 within the body conforming layer 38. Each slit 50 within the body conforming layer 38 can be completely surrounded by non-slitted absorbent material forming the body conforming layer 38. Each slit 50 can extend in the longitudinal direction (X) of the body conforming layer 38 such that each slit 50 can have a length in the longitudinal direction (X) which is greater than a width of the slit 50 in the transverse direction (Y). Each slit 50 can have a length in the longitudinal direction (X) from about 2, 3, 5, 7, 10, 12, or 15 mm to about 20, 25, 30, or 35 mm. A width of the slit 50 in the transverse direction (Y) is measured as the distance in the transverse direction between the absorbent materials of the body conforming layer 38 which form the perimeter of the slit 50. In various embodiments, each slit 50 can be formed by cutting the absorbent material of the body conforming layer 38 such that each slit 50 has less than 1.5, 1, or 0.5 mm width in the transverse direction (Y). In various embodiments, each slit 50 can be formed by cutting the absorbent material of the body conforming layer 38 such that each slit 50 has 0 mm width in the transverse direction (Y) of the body conforming layer 38. In various embodiments, while no absorbent material is removed from the body conforming layer 38 during the formation of each slit 50 some absorbent material adjacent each slit 50 may be deformed during the formation of the slit 50 thereby resulting in a minimal width in the transverse direction (Y) of the body conforming layer 38.

In various embodiments, each individual slit 50 may have a shape such as, for example, a straight line, an arcuate shape, S-shape, V-shape, Z-shape, U-shape, or any other suitable shape. In various embodiments, each slit 50 within the body conforming layer 38 can have the same shape. In various embodiments, a slit 50 within the body conforming layer 38 can have a shape which is different from another slit 50 within the body conforming layer 38.

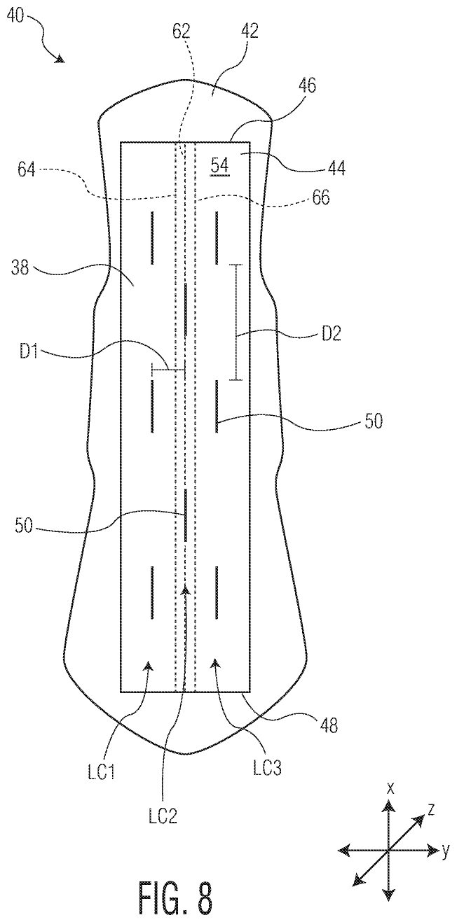

In various embodiments, the slits 50 within a body conforming layer 38 can be incorporated into the body conforming layer 38 in a random arrangement but where each slit 38 is situated at a distance from another slit 50. In various embodiments, the slits 50 within a body conforming layer 38 can be incorporated in a pattern arrangement. In various embodiments, at least one longitudinal direction column(s) LC of slits 50 can be formed into the body conforming layer 38, such as, for example, illustrated in FIG. 8 wherein three longitudinal direction columns, LC1, LC2, and LC3, are illustrated. In various embodiments, at least 1, 2, 3, 4, 5, 6, 7, 8, 9, or 10 longitudinal direction columns LC of slits 50 can be formed into the body conforming layer 38. In various embodiments, the body conforming layer 38 can have 1, 2, 3, 4, 5, 6, 7, 8, 9, or 10 longitudinal direction columns of slits 50. Each longitudinal direction column LC can be separated in the transverse direction (Y) from the next adjacent longitudinal direction column LC by a distance D1 from about 3, 5, 7, or 9 mm to about 12, 15, 17, or 20 mm.

Each longitudinal direction column LC can have at least 1, 2, 3, 4, 5, 6, 7, 8, 9, or 10 slits 50 extending in the longitudinal direction (X) within the same longitudinal direction column LC. As illustrated in FIG. 8, three longitudinal direction columns, LC1, LC2, and LC3, are illustrated wherein LC1 and LC3 each have three slits 50 extending in the longitudinal direction (X) and LC2 has two slits 50 extending in the longitudinal direction (X). Each slit 50 in a longitudinal direction column LC can be separated in the longitudinal direction (X) from the next adjacent slit 50 within the same longitudinal direction column LC by a distance D2 from about 5, 10, or 15 mm to about 20, 25, 30, or 35 mm.

In various embodiments in which at least two longitudinal direction columns, such as LC1 and LC2, of slits 50 are incorporated into the body conforming layer 38, the slits 50 can be incorporated into the body conforming layer 38 such that the slits of longitudinal direction column LC1 are offset in the transverse direction (Y) from the slits 50 of the next adjacent longitudinal direction column LC2. FIG. 8 provides an illustration in which the slits 50 of longitudinal direction column LC1 are offset in the transverse direction (Y) from the slits 50 of longitudinal direction column LC2. In such an illustration, an imaginary transverse direction line placed perpendicular to a slit 50 of longitudinal direction column LC1 will not intersect a slit 50 of longitudinal direction column LC2.

In various embodiments in which at least two longitudinal direction columns, such as LC1 and LC2, of slits are incorporated into the body conforming layer 38, the slits 50 can be incorporated into the body conforming layer 38 such that a slit 50 of longitudinal direction column LC1 at least partially overlaps in the transverse direction (Y) with a slit 50 of the next adjacent longitudinal direction column LC2. In such embodiments, an imaginary transverse direction line placed perpendicular to a slit 50 in longitudinal direction column LC1 will intersect a slit 50 of the next adjacent longitudinal direction column LC2. In various embodiments in which a slit 50 from a longitudinal direction column LC1 at least partially overlaps with a slit 50 in the next adjacent longitudinal direction column LC2, the amount of overlap is less than about 75, 60, 50, 40, 30, 25, 20, 15, or 10% of the longitudinal direction (X) length of each slit 50.

In addition to the slits 50, the body conforming layer 38 can have a crease 60 extending in the longitudinal direction (X) of the body conforming layer 38. The crease 60 can be formed by incorporating fold lines, 62, 64 and 66, into the body conforming layer 38. When the absorbent article 10 is in use, the pressure exerted on the absorbent article 10 by the movement of the wearer 10 can cause the fold lines, 62, 64, and 66, to bend resulting in the release of stress on the body conforming layer 38. The bending of each of the fold lines, 62, 64, and 66, can force the apex of the crease 60, such as formed by the center fold line such as, for example, fold line 62 in the Figures, higher in elevation towards the topsheet layer 30 of the absorbent article 10. When the pressure exerted on the absorbent article 10 is released, the fold lines, 62, 64, and 66, can unfold and the apex of the crease 60 can lower away from the topsheet layer 30 of the absorbent article 10. The crease 60 can extend from the first transverse direction end edge 46 of the body conforming layer 38 to the second transverse direction end edge 48 of the body conforming layer 38. The crease 60 can be centered along the longitudinal centerline 26 of the absorbent article 10. In various embodiments, at least one slit 50 in the body conforming layer 38 can overlap the crease 60, such as, for example, illustrated in the Figures, wherein the slits 50 of longitudinal direction column LC2 overlap fold line 62 of crease 60 at the longitudinal centerline 26 of the absorbent article 10. In various embodiments, the body conforming layer 38 can have more than one crease 60.

In various embodiments, a layer of the absorbent assembly 40, whether the body conforming layer 38 or another layer of the absorbent assembly 40, can generally be any single layer structure or combination of layer components, which can demonstrate some level of compressibility, conformability, be non-irritating to the wearer's skin, and capable of absorbing and retaining liquids and other body exudates. In various embodiments, a layer of the absorbent assembly 40 can be formed from a variety of different materials and can contain any number of desired layers. For example, a layer of the absorbent assembly 40 can include one or more layers (e.g., two layers) of absorbent web material of cellulosic fibers (e.g., wood pulp fibers), other natural fibers, synthetic fibers, woven or nonwoven sheets, scrim netting, or other stabilizing structures, superabsorbent material, binder materials, surfactants, selected hydrophobic and hydrophilic materials, pigments, lotions, odor control agents or the like, as well as combinations thereof. In an embodiment, the absorbent web material can include a matrix of cellulosic fluff and can also include superabsorbent material. The cellulosic fluff can comprise a blend of wood pulp fluff. An example of wood pulp fluff can be identified with the trade designation NB416, available from Weyerhaeuser Corp., and is a bleached, highly absorbent wood pulp containing primarily soft wood fibers.

In various embodiments, if desired, the absorbent assembly 40 can include an optional amount of superabsorbent material. Examples of suitable superabsorbent material can include poly(acrylic acid), poly(methacrylic acid), poly(acrylamide), poly(vinyl ether), maleic anhydride copolymers with vinyl ethers and α-olefins, poly(vinyl pyrrolidone), poly(vinylmorpholinone), poly(vinyl alcohol), and salts and copolymers thereof. Other superabsorbent materials can include unmodified natural polymers and modified natural polymers, such as hydrolyzed acrylonitrile-grafted starch, acrylic acid grafted starch, methyl cellulose, chitosan, carboxymethyl cellulose, hydroxypropyl cellulose, and natural gums, such as alginates, xanthan gum, locust bean gum, and so forth. Mixtures of natural and wholly or partially synthetic superabsorbent polymers can also be useful. The superabsorbent material can be present in the absorbent assembly 40 in any amount as desired.

Regardless of the combination of absorbent materials used in a layer of the absorbent assembly 40, the absorbent materials for each layer of the absorbent assembly 40 can be formed into a web structure by employing various conventional methods and techniques. For example, the absorbent web can be formed by techniques such as, but not limited to, a dry-forming technique, an air forming technique, a wet forming technique, a foam forming technique, or the like, as well as combinations thereof. A coform nonwoven material can also be employed. Methods and apparatus for carrying out such techniques are well known in the art.

The overall shape of the absorbent assembly 40 can vary as desired and can comprise any one of various shapes including, but not limited to, triangular, rectangular, dog-bone, elliptical, trapezoidal, T-shape, I-shape, and hourglass shapes. In various embodiments, the absorbent assembly 40 can have a shape that generally corresponds with the overall shape of the absorbent article 10. The dimensions of the absorbent assembly 40 can be substantially similar to those of the absorbent article 10, however, it will be appreciated that the dimensions of the absorbent assembly 40 while similar, will often be less than those of the overall absorbent article 10, in order to be adequately contained therein. The size and the absorbent capacity of the absorbent assembly 40 should be compatible with the size of the intended wearer and the liquid loading imparted by the intended use of the absorbent article 10.

In various embodiments in which the absorbent assembly 40 contains a single layer which is a body conforming layer 38, the overall shape of the body conforming layer 38 can vary as desired and can comprise any one of various shapes including, but not limited to, triangular, rectangular, dog-bone, elliptical, trapezoidal, T-shape, I-shape, and hourglass shapes. In various embodiments, the body conforming layer 38 can have a shape that generally corresponds with the overall shape of the absorbent article 10. The dimensions of the body conforming layer 38 can be substantially similar to those of the absorbent article 10, however, it will be appreciated that the dimensions of the body conforming layer 38 while similar, will often be less than those of the overall absorbent article 10, in order to be adequately contained therein. The size and the absorbent capacity of the body conforming layer 38 should be compatible with the size of the intended wearer and the liquid loading imparted by the intended use of the absorbent article 10.

In various embodiments in which the absorbent assembly 40 has an upper absorbent layer 42 and a lower absorbent layer 44, the overall shape of each of the upper absorbent layer 42 and lower absorbent layer 44 of the absorbent assembly 40 can vary as desired and can comprise any one of various shapes including, but not limited to, triangular, rectangular, dog-bone, elliptical, trapezoidal, T-shape, I-shape, and hourglass shapes. In various embodiments, each of the upper absorbent layer 42 and lower absorbent layer 44 of the absorbent assembly 40 can have a shape that generally corresponds with the overall shape of the absorbent article 10. In various embodiments, the upper absorbent layer 42 can have a shape that generally corresponds to the overall shape of the absorbent article 10 while the lower absorbent layer 44 can have a different shape, such as, for example, a rectangle. The dimensions of each of the upper absorbent layer 42 and lower absorbent layer 44 of the absorbent assembly 40 can be substantially similar to those of the absorbent article 10, however, it will be appreciated that the dimensions of the upper absorbent layer 42 and the lower absorbent layer 44 of the absorbent assembly 40 while similar, will often be less than those of the overall absorbent article 10, in order to be adequately contained therein. In various embodiments, the upper absorbent layer 42 can have a size dimension which can be larger than the size dimension of the lower absorbent layer 44. The size and the absorbent capacity of each of the upper absorbent layer 42 and lower absorbent layer 44 of the absorbent assembly 40 should be compatible with the size of the intended wearer and the liquid loading imparted by the intended use of the absorbent article 10.

In various embodiments, an absorbent assembly 40 can have at least two layers of material, such as, for example, an upper absorbent layer 42 and a lower absorbent layer 44. In various embodiments, the upper absorbent layer 42 and the lower absorbent layer 44 can be identical to each other in configuration of absorbent materials, size dimension, and shape. In various embodiments, the upper absorbent layer 42 and the lower absorbent layer 44 can be different from each other in at least one of configuration of absorbent materials, size dimension, and shape. In various embodiments, the upper absorbent layer 42 and the lower absorbent layer 44 can be different from each other in at least two of configuration of absorbent materials, size dimension, and shape. In various embodiments, the upper absorbent layer 42 and the lower absorbent layer 44 can be different from each other in each of configuration of absorbent materials, size dimension, and shape. In various embodiments, the upper absorbent layer 42 and the lower absorbent layer 44 can provide the absorbent article 10 with different absorption properties as deemed suitable. As an example of an absorbent assembly 40, in various embodiments, the upper absorbent layer 42 of the absorbent assembly 40 may be constructed of a coform material and the lower absorbent layer 44 of the absorbent assembly 40 may be constructed of a superabsorbent polymer-containing compressed sheet. In such embodiments, the coform material can have a basis weight from about 20, 30, or 40 to about 200, 400, or 600 gsm and the superabsorbent polymer-containing compressed sheet can be a cellulosic fluff based material that can be a combination of cellulosic pulp and SAP enclosed with a tissue carrier and having a basis weight from about 40 to about 400 gsm. The coform upper absorbent layer 42 can have a shape generally corresponding to the shape of the absorbent article 10 while the superabsorbent polymer-containing compressed sheet lower absorbent layer 44 can have a rectangular shape and can be of a size dimension smaller than the coform upper absorbent layer 42.

By way of example, suitable materials and/or structures for a layer of the absorbent assembly 40 can include, but are not limited to, those described in U.S. Pat. No. 4,610,678 to Weisman, et al., U.S. Pat. No. 6,060,636 to Yahiaoui, et al., U.S. Pat. No. 6,610,903 to Latimer, et al., U.S. Pat. No. 7,358,282 to Krueger, et al., and U.S. Publication No. 2010/0174260 to Di Luccio, et al. each of which is hereby incorporated by reference thereto in its entirety.

Liquid Impermeable Layer:

The liquid impermeable layer 36 is generally liquid impermeable and is the portion of the absorbent article 10 which faces the garments of the wearer. The liquid impermeable layer 36 can permit the passage of air or vapor out of the absorbent article 10 while still blocking the passage of liquids. Any liquid impermeable material may generally be utilized to form the liquid impermeable layer 36. The liquid impermable layer 36 can be composed of a single layer or multiple layers, and these one or more layers can themselves comprise similar or different materials. Suitable material that may be utilized can be a microporous polymeric film, such as a polyolefin film or polyethylene or polypropylene, nonwovens, and nonwoven laminates, and film/nonwoven laminates. The particular structure and composition of the liquid impermeable layer 36 can be selected from various known films and/or fabrics with the particular material being selected as appropriate to provide the desired level of liquid barrier, strength, abrasion resistance, tactile properties, aesthetics, and so forth. In various embodiments, a polyethylene film can be utilized that can have a thickness in the range of from about 0.2 or 0.5 mils to about 3.0 or 5.0 mils. An example of a liquid impermeable layer 36 can be a polyethylene film such as that obtainable from Pliant Corp., Schaumburg, Ill., USA. Another example can include calcium carbonate-filled polypropylene film. In still another embodiment, the liquid impermeable layer 36 can be a hydrophobic nonwoven material with water barrier properties such as a nonwoven laminate, an example of which can be a spunbond, meltblown, meltblown, spunbons, four-layered laminate.

The liquid impermeable layer 36 can, therefore, be of a single or multiple layer construction, such as of multiple film layers or laminates of film and nonwoven fibrous layers. Suitable liquid impermeable layers 36 can be constructed from materials such as those described in U.S. Pat. No. 4,578,069 to Whitehead, et al., U.S. Pat. No. 4,376,799 to Tusim, et al., U.S. Pat. No. 5,695,849 to Shawver, et al., U.S. Pat. No. 6,075,179 to McCormack, et al., and U.S. Pat. No. 6,376,095 to Cheung, et al., each of which are hereby incorporated by reference thereto in its entirety.

Fluid Intake Layer:

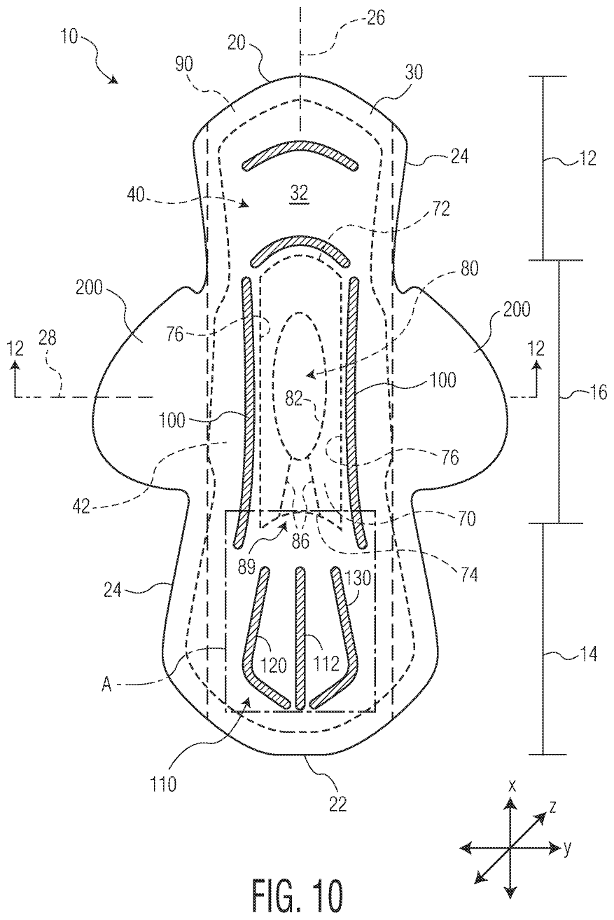

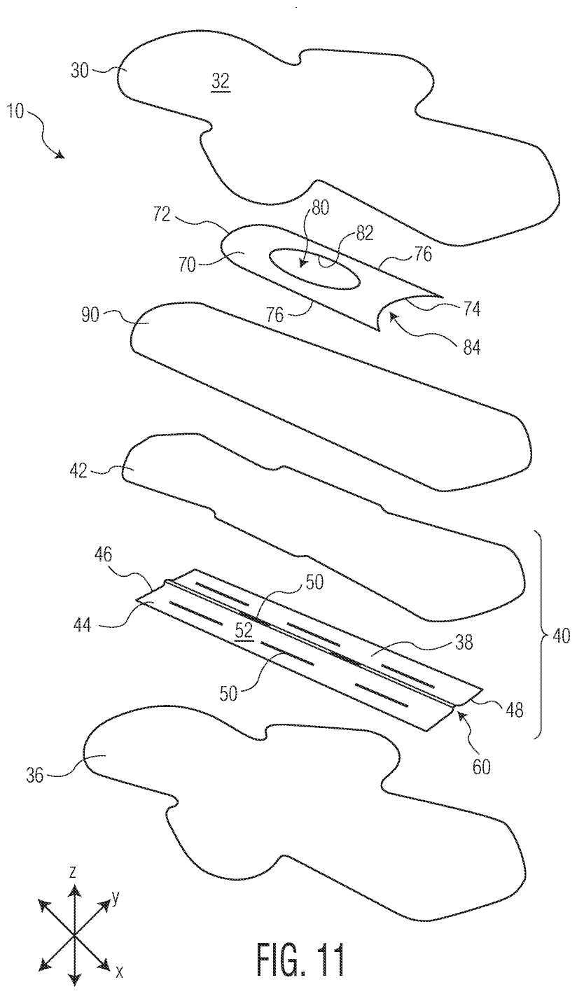

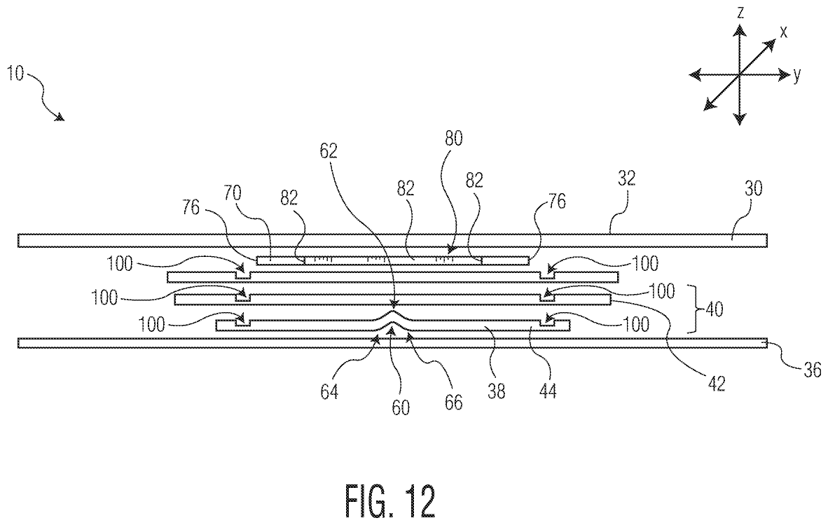

In various embodiments, the absorbent article 10 can include a liquid permeable fluid intake layer 70 positioned between the topsheet layer 30 and the absorbent assembly 40. For example, FIGS. 10-12 provide an exemplary illustration of a fluid intake layer 70 positioned between the topsheet layer 30 and the absorbent assembly 40 of an absorbent article 10. The fluid intake layer 70 can have a first transverse direction end edge 72, a second transverse direction end edge 74, and an opposing pair of longitudinal direction side edges 76 extending between and connecting the transverse direction end edges, 72 and 74. In various embodiments, the first transverse direction end edge 72 can be the leading edge of the fluid intake layer 70 closest to the first transverse direction end edge 20 of the absorbent article 10. In various embodiments, the second transverse direction end edge 74 of the fluid intake layer 70 can be the trailing edge of the fluid intake layer 70 closest to the second transverse direction end edge 22 of the absorbent article 10.

A fluid intake layer 70 can be made of a material that can be capable of rapidly transferring, in the depth direction (Z), body exudates that are delivered to the topsheet layer 30. The fluid intake layer 70 can generally have any shape and/or size desired. In an embodiment, the fluid intake layer 70 can have a curved rectangular shape, with a length equal to or less than the overall length of the absorbent article 10, and a width equal to or less than the width of the absorbent article 10. For example, the fluid intake layer 70 can have a longitudinal length of between about 20, 40, 60, 70, 80, or 90 mm to about 100, 110, 120, 130, 140, or 150 mm and a transverse width of between about 10, 15, 20, or 30 mm to about 35, 40, 45, 50, or 60 mm may be utilized. The fluid intake layer 70 can have a height in the depth direction (Z) from about 0.5 mm to about 3 mm. The fluid intake layer 70 can have a basis weight from about 10, 25, or 100 gsm to about 200, 250, or 300 gsm. In various embodiments, the fluid intake layer 70 can have a longitudinal length that is from about 25 or 40% to about 50 or 60% of the longitudinal length of the absorbent article 10. In various embodiments, the fluid intake layer 70 can have a transverse width that is from about 15, 20, 25 or 30% to about 50, 55, 60, or 70% of the transverse width of the absorbent article 10.

Any of a variety of different materials can be capable of being used for the fluid intake layer 70 to accomplish the above-mentioned functions. The material may be synthetic, cellulosic, or a combination of synthetic and cellulosic materials. Alternatively, aperture films, such as those available from such film suppliers as Texol, Italy and Tredegar, U.S.A. may be utilized. The fluid intake layer 70 can be constructed from any woven or nonwoven material. For example, the fluid intake layer 70 can be constructed as an airlaid, spunbond, tissue, meltblown, spunbond-meltblown-spunbond, or TABCW material. For example, airlaid cellulosic tissues may be suitable for use in the fluid intake layer 70. The airlaid cellulosic tissue may have a basis weight ranging from about 10 or 100 gsm to about 250 or 300 gsm. The airlaid cellulosic tissue can be formed from hardwood and/or softwood fibers. An airlaid cellulosic tissue can have a fine pore structure and can provide an excellent wicking capacity, especially for menses. In various embodiments, fluid intake layer 70 can be a TABCW material laminated with an airlaid material where the TABCW/airlaid laminate has a basis weight of 30, 50, 75, 100, or 125 gsm to 150, 168, 200, 250, or 300 gsm, a height of 0.5, 1, or 1.7 mm to 2.0, 2.8, 3.5, or 4 mm, and a density of about 0.06 g/cc. The TABCW portion of this laminate may include polyethylene fibers, polypropylene fibers, polyester fibers, polyethylene/polypropylene bicomponent fibers, or combinations thereof. The airlaid portion of this laminate may include fluff pulp fibers and about 15-20% bicomponent fibers by weight. In various embodiments, a fluid intake layer 70 can be a coform material having a basis weight of 157 to 202 gsm, a thickness of 2.2 to 2.6 mm, and a density of about 0.08 g/cc. This coform material may include fluff pulp fibers and about 31% bicomponent fibers by weight. The bicomponent fibers may be polypropylene fibers. This coform material may be formed on a spunbond carrier sheet. In various embodiments, the fluid intake layer 70 can be a thermally bonded web having a basis weight of about 125 to about 160 gsm, a thickness of 2.25 mm to 2.75 mm, and a density of about 0.006 g/cc. This thermally bonded material may include fluff pulp fibers and about 21% bicomponent fibers by weight. The bicomponent fibers may be polypropylene fibers. In various embodiments, the fluid intake layer 70 can be at least partially hydrophilic. In various embodiments, the hydrophilicity of the fluid intake layer 70 can be increased or created via treatment of the fluid intake layer 70 with surfactants.

In various embodiments, a foam material can be utilized to form the fluid intake layer 70. In various embodiments, the foam material can be an open-cell or porous foam. The physical properties of the foam material as well as its wettability and fluid management properties can be tailored to meet the specific characteristics desired for the usage of a foam material in the absorbent article 10. In various embodiments, the foam material can be moisture stable and not degrade or collapse and lose its structure and fluid management properties when exposed to body exudate. In various embodiments, the foam material can be an open-cell foam, a closed cell foam, or a partially open-cell foam that is either a thermoplastic or thermoset material. A foam material can be manufactured by extrusion or casting and coating processes including frothed foam, aerated foam, and emulsion foam methods. Such foams can be manufactured from different polymer chemistries to achieve the desired softness, flexibility, and resilience of the foam material when utilized in an absorbent article 10. In various embodiments, the foam material can be based on organic or inorganic chemistries and can also be based upon a foam material obtained from natural sources. In various embodiments, the foam material can have a polymer chemistry which can be a polyurethane foam, polyolefin foam, poly(styrene-butadiene) foam, poly(ethylene-vinyl acetate) foam, or a silicone based foam. Other polymer chemistries known to one of ordinary skill in the art could be used along with additives such as plasticizers, opacifiers, colorants, antioxidants, and stabilizers to obtain the desired foam properties. In various embodiments, the viscoelastic properties could be modified to obtain a desired response to applied load from the foam material including properties similar to that commonly referred to as polyurethane memory foam materials. In various embodiments, the Poisson's ratio of the foam material could be modified to obtain the desired response from the foam material to applied stress and foam materials with auxetic properties could be considered if desired.

Additionally, to further enhance the ability of the absorbent article 10 to transfer body exudates in the depth direction (Z) from the topsheet layer 30 toward any lower layers in the absorbent article 10 as well as to enhance the ability of the fluid intake layer 70 to conform to the wearer's body based on its ability to bend, the fluid intake layer 70 can have an opening 80 in the fluid intake layer 70 which can be any suitable shape, such as ovular, circular, rectangular, square, triangular, etc. In various embodiments, the opening 80 in the fluid intake layer 70 can be elongate and can be oriented in the longitudinal direction (X) of the absorbent article 10. The opening 80 in the fluid intake layer 70 can be bounded by a perimeter 82 which can form an inner edge of the fluid intake layer 70. The opening 80 can form a cup or well-like structure for holding body exudates and preventing its leakage away from a region of the absorbent article 10 and towards the edges of the absorbent article 10.

The opening 80 can be located at various positions along the longitudinal and transverse directions of the fluid intake layer 70 depending upon the primary location of body exudate intake or the purpose for which the absorbent article 10 is being used. For example, in various embodiments, the fluid intake layer 70 and the opening 80 can be positioned so that they are in substantial alignment with the longitudinal centerline 26 and the transverse centerline 28 of the absorbent article 10. This allows the opening 80 to be centrally disposed so that it can be positioned below the main point of body exudate discharge and so that it can act as the primary body exudate receiving area for the absorbent article 10.

However, centralized positioning of the fluid intake layer 70 and the opening 80 is not required, and in various embodiments, depending on the primary location where body exudate intake might occur, the fluid intake layer 70 and the opening 80 may be substantially aligned with the longitudinal centerline 26 only. Thus, in various embodiments, the fluid intake layer 70 and the opening 80 may be shifted in the longitudinal direction (X) towards either transverse direction end edge, 20 or 22, of the absorbent article 10, so that the opening 80 is not in substantial alignment with the transverse centerline 28.

The opening 80 can have a longitudinal length from about 15, 20, 30, or 40 mm to about 50, 60, 70, or 75 mm and can have a transverse width from about 10, 15, or 20 mm to about 25, 30, 35, or mm. The opening 80 can have a length that is from about 15, 20, or 25% to about 70, 75, or 80% of the overall longitudinal length of the fluid intake layer 70 in the longitudinal direction (X). The opening 80 can have a width that can be from about 20, 25, or 30% to about 70, 75, or 80% of the overall width of the fluid intake layer 80 in the transverse direction (Y).

Additionally, the fluid intake layer 70 has a rearward-facing arch 84 located along the second transverse direction end edge 74 of the fluid intake layer 70. The rearward-facing arch 84 is believed to enhance the ability of the absorbent article 10 to form an upward tented configuration. The rearward-facing arch 84 is also believed to enhance the ability of the fluid intake layer 70 to conform to the wearers body based upon its ability to bend.

In various embodiments, the fluid intake layer 80 may further include one or more haunches 86. The haunches 86 may be created by embossing, perforating, bonding, scoring, or otherwise manipulating the fluid intake layer 70. The haunches 86 are believed to improve body conformance. The haunches 86 may also work in conjunction with other structure of the absorbent article 10 to help initiate and/or maintain the tented configuration of the absorbent article 10. The haunches 86 may be generally aligned with one or more flexures as described below.

Flexures:

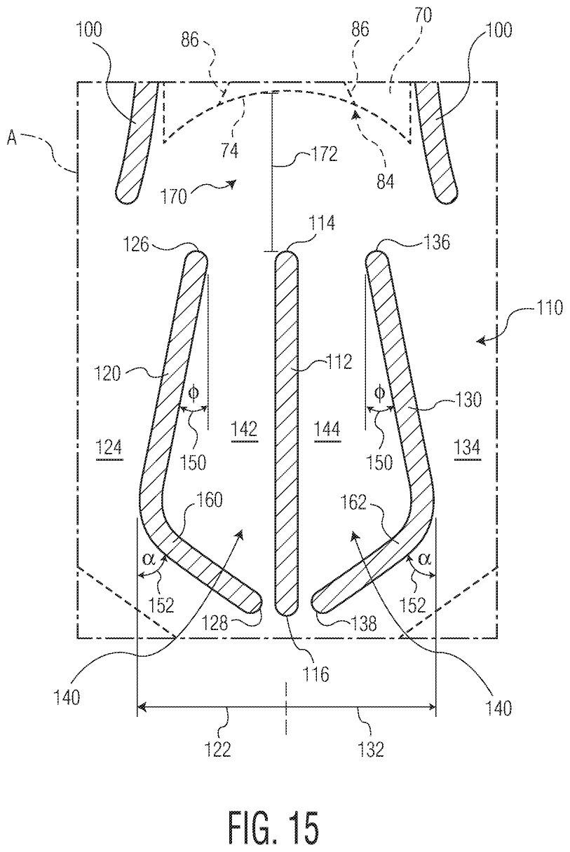

The absorbent article 10 can have one or more flexures 110 positioned in the posterior region 14 of the absorbent article 10 such as, for example, illustrated in FIGS. 1 and 10. Additionally, details of the flexures 110 can be seen in FIG. 15 which is a magnified view of the area of FIG. 10 bound by the dot-dash rectangle A. In addition to the slits 50 and crease 60, flexures 110 can help influence the shaping and folding of the absorbent article 10 into a tented configuration in the posterior region 14 of the absorbent article 10. The flexures 110 can be created by physical discontinuities in the absorbent article 10 and/or elements in the absorbent article 10. For example, the flexures 110 can be created by pre-folding, scoring, indenting, perforating, embossing, bonding, or combinations thereof. In various embodiments, the flexures 110 can be created by scoring, folding, indenting, perforating, embossing, or bonding one or more layers of the absorbent assembly 40. Flexures 110 may also be created with changes in elevation and/or density in the absorbent article 10 and/or elements of the absorbent article 10. In various embodiments, the flexures 110 can help regulate dynamic movement of the posterior region 14 of the absorbent article 10.

In various embodiments, the flexures 110 of the absorbent article 10 can include a first flexure 112 in the posterior region 14 of the absorbent article 10. The first flexure 112 may extend in a direction generally parallel to the longitudinal centerline 26. The first flexure 112 defines a front point 114 and a rear point 116.

In various embodiments, the flexures 110 of the absorbent article 10 can include a second flexure 120 in the posterior region 14 of the absorbent article 10. The second flexure 120 can be spaced transversely outward from the first flexure 112 in a first direction 122. The second flexure 120 defines a first side portion 124 of the absorbent article 10. The first side portion 124 is positioned transversely outward from the second flexure 120 in the first direction 122 such as illustrated in FIG. 15. The second flexure 120 defines a front point 126 and a rear point 128.

In various embodiments, the flexures 110 of the absorbent article 10 can include a third flexure 130 in the posterior region 14 of the absorbent article 10. The third flexure 130 can be spaced transversely outward from the first flexure 112 in the second direction 132. The second direction 132 is opposite the first direction 122. The third flexure 130 defines a second side portion 134 of the absorbent article 10. The second side portion 134 is positioned laterally outward from the third flexure 130 in the second direction 132 such as illustrated in FIG. 15. The third flexure 130 defines a front point 136 and a rear point 138.

The second flexure 120 and the third flexure 130 define a dynamic region 140 of the absorbent article 10 therebetween. The second flexure 120 and the first flexure 112 define a first side 142 of the dynamic region 140. The third flexure 130 and the first flexure 112 define a second side 144 of the dynamic region 140.

In various embodiments, the second flexure 120 and/or the third flexure 130 may be oriented in a direction generally parallel to the longitudinal centerline 26. In various embodiments, the second flexure 120 and/or the third flexure 130 may be positioned at an angle relative to the longitudinal centerline 26. For example, in various embodiments, the second flexure 120 and/or the third flexure 130 may diverge rearward at a first angle 150 relative to the longitudinal centerline 26. In various embodiments, the first angle 150 may be from about 1, 2, or 3 degrees to about 5, 7, or 10 degrees relative to the longitudinal centerline 26. In various embodiments, the second flexure 120 and/or the third flexure 130 may be positioned at an angle relative to the longitudinal centerline 26 and may be generally aligned with one or more haunches 86. For example, both the second flexure 120 and the third flexure 130 are aligned with the haunches 86 as illustrated in FIG. 15.

In various embodiments, the second flexure 120 may include a first return portion 160 converging rearward at a second angle 152 relative to the longitudinal centerline 26. In various embodiments, the third flexure 130 may include a second return portion 162 converging rearward at the second angle 152 relative to the longitudinal centerline 26. The first return portion 160 and the second return portion 162 can help to form the tented configuration in the anterior region 14 of the absorbent article 10 by helping transfer lateral compression into the upward tented configuration.

In various embodiments, the first flexure 112, the second flexure 120, and the third flexure 130 are embossments in the absorbent assembly 40. In various embodiments, the first flexure 112, the second flexure 120, and the third flexure 130 are embossments in the upper absorbent layer 42 of the absorbent assembly 40.

In various embodiments, the first flexure 112, the second flexure 120, and/or the third flexure 130 extend under the fluid intake layer. In various embodiments, the first flexure 112, the second flexure 120, and/or the third flexure 130 terminate at respective front points 114, 126, and 136 proximate the rearward-facing arch 84 of the fluid intake layer 70. The longitudinal spacing between the respective front points 114, 126, and 136 and the rearward-facing arch 70 defines a transition zone 170 having a transition zone length 172. In various embodiments, the transition zone 170 provides a region of lower density as compared to the flexures 110 and the fluid intake layer 70.

Fluid Distribution Layer:

In various embodiments, the absorbent article 10 can have a fluid distribution layer 90. FIGS. 10-12 provide an exemplary illustration of a fluid distribution layer 90 positioned between the fluid intake layer 70 and the absorbent assembly 40 of the absorbent article 10. In various embodiments, the fluid distribution layer 90 can be positioned between the fluid intake layer 70 and the topsheet layer 30 of the absorbent article 10. The fluid distribution layer 90 can help decelerate and diffuse surges or gushes of liquid body exudates penetrating the topsheet layer 30. The fluid distribution layer 90 may have any longitudinal length dimension as deemed suitable. In an embodiment, the longitudinal length of the fluid distribution layer 90 can be the same as the longitudinal length of the absorbent assembly 40. In an embodiment, the longitudinal length of the fluid distribution layer 90 can be shorter than the longitudinal length of the absorbent assembly 40. In such an embodiment, the fluid distribution layer 90 may be positioned at any desired location along the longitudinal length of the absorbent assembly 40. As an example of such an embodiment, the absorbent article 10 may contain a target area where repeated liquid surges typically occur in the absorbent article 10.

In various embodiments, the fluid distribution layer 90 can include natural fibers, synthetic fibers, superabsorbent material, woven material, nonwoven material, wet-laid fibrous webs, a substantially unbounded airlaid fibrous web, an operatively bonded, stabilized-airlaid fibrous web, or the like, as well as combinations thereof. Alternatively, aperture films, such as those available from such film suppliers as Texol, Italy and Tredegar, U.S.A. may be utilized. In various embodiments, the fluid distribution layer 90 can be formed from a material that is substantially hydrophobic, such as a nonwoven web composed of polypropylene, polyethylene, polyester, and the like, and combinations thereof. In various embodiments, the fluid distribution layer 90 can include conjugate, biconstituent, and/or homopolymer fibers of staple or other lengths and mixtures of such fibers with other types of fibers. In various embodiments, the fluid distribution layer 90 can have fibers which can have a denier of greater than about 5. In various embodiments, the fluid distribution layer 90 can have fibers which can have a denier of less than about 5. In various embodiments, the fluid distribution layer 90 can be a bonded carded web or an airlaid web. In various embodiments, the bonded carded web may be, for example, a powder bonded carded web, an infrared bonded carded web, or a through air bonded carded web.

In various embodiments, the basis weight of the fluid distribution layer 90 can be at least about 10 or 20 gsm. In various embodiments, the basis weight of the fluid distribution layer 90 can be from about 10, 20, 30, 40, 50 or 60 gsm to about 65, 70, 75, 80, 85, 90, 100, 110, 120, or 130 gsm. In various embodiments, the basis weight of the fluid distribution layer 90 can be less than about 130, 120, 110, 100, 90, 85, 80, 75, 70, 65, 60 or 50 gsm.

Lateral Stiffeners:

In various embodiments, the absorbent article can have one or more lateral stiffeners 100. The lateral stiffeners 100 can be positioned within the central region 16 of the absorbent article 10 such as, for example, illustrated in FIG. 1. In various embodiments, the lateral stiffeners 100 can be applied along the longitudinal direction side edges 76 of the fluid intake layer 70 such as, for example, illustrated in FIG. 10. The lateral stiffeners 100 positioned along the longitudinal direction side edges 76 of a fluid intake layer 70 can transfer compressive forces applied by the legs of the wearer at the central region 16 of the absorbent article 10 to other regions of the absorbent article 10 where tenting is desired. For example, the lateral stiffeners 100 can transfer compressive forces applied by the legs of the wearer at the central region 16 to both the anterior region 12 and the posterior region 14 of the absorbent article 10 wherein dynamic conformance to the wearer's body is desired.

The lateral stiffeners 100 can be produced by added a stiff material, embossing, folding, pleating, bonding, and the like, and combinations thereof. In various embodiments, the lateral stiffeners 100 may be produced by embossing one or more elements of the absorbent article 10, such as the absorbent assembly 40. Likewise, the lateral stiffeners 100 can be produced by applying lines of hot melt adhesive or plastic strips. The lateral stiffeners 100 may be created by folding one or more elements of the absorbent article 10, such as the topsheet layer 30 and liquid impermeable layer 36. Stiff attachment means, such as hook and loop fastener strips, may act as lateral stiffeners or may add stiffness to the lateral stiffeners 100.

Fastening System:

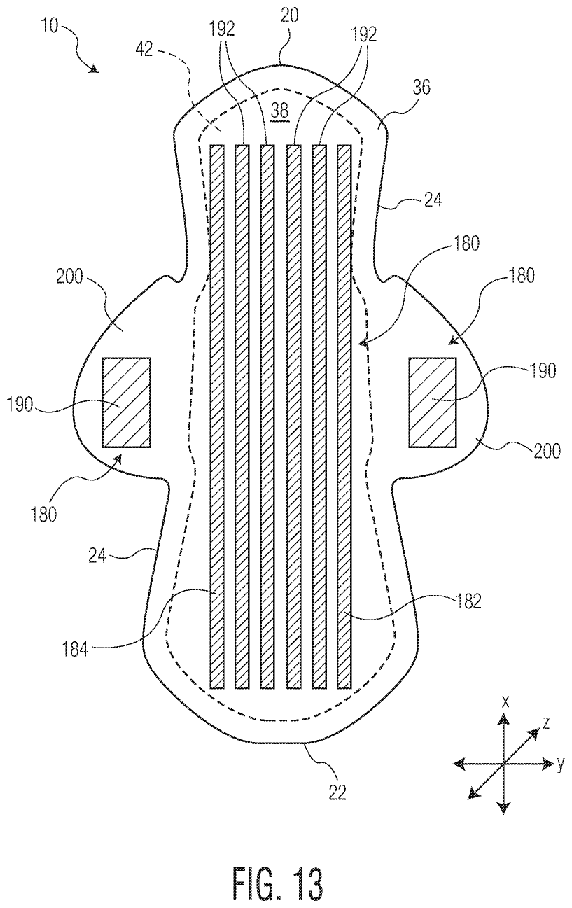

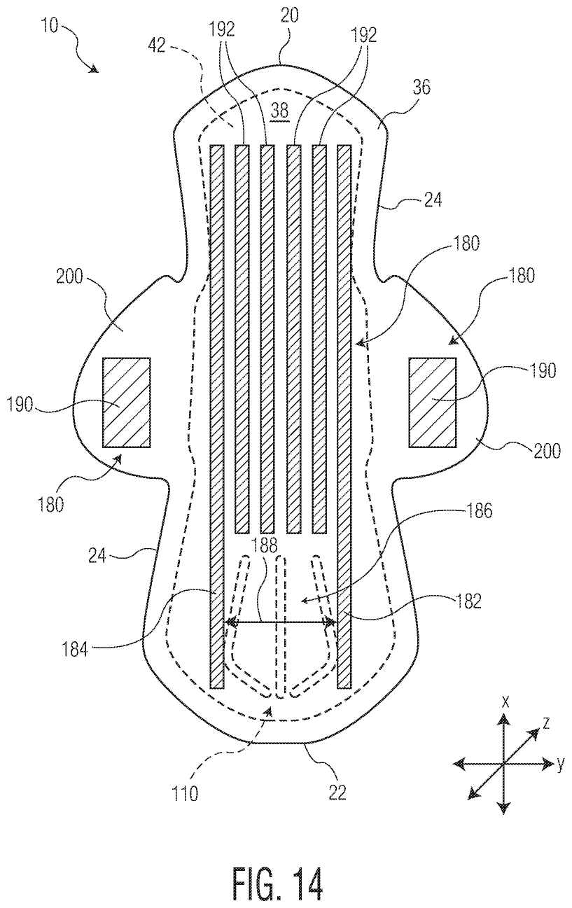

In various embodiments, the absorbent article 10 includes a fastening system 180 such as, for example, illustrated in FIGS. 13 and 14. The fastening system 180 can be adapted to secure the absorbent article 10 to the undergarment of the wearer. In various embodiments, the fastening system 180 can include one or more separate fasteners positioned in any suitable arrangement on the garment facing surface 38 of the liquid-impermeable layer 36. In various embodiments, such as, for example, illustrated in FIG. 13, the fastening system 180 can include a first fastener 182 attached to the garment facing surface 38 of the liquid impermeable layer 36 in the first side portion 142, a second fastener 184 attached to the garment facing surface 38 of the liquid impermeable layer 36 in the second side portion 144, and a plurality of additional fasteners 192 positioned between the first fastener 182 and the second fastener 184. Each of the first fastener 182, the second fastener 184, and the plurality of fasteners 192 positioned therebetween can extend from the posterior region 14 and through the central region 16 to the anterior region 12 of the absorbent article 10. In various embodiments, such as, for example, illustrated in FIG. 14, the fasting system 180 can include a first fastener 182 attached to the garment facing surface 38 of the liquid impermeable layer 36 in the first side portion 142 and a second fastener 184 attached to the garment facing surface 38 of the liquid impermeable layer 36 in the second side portion 144. In various embodiments, the central portion 186 of the garment facing surface 38 of the liquid impermeable layer 36 is substantially free of fasteners in the posterior region 14. In various embodiments, the central portion 186 may have a width 188 in the transverse direction (Y) of 15, 20, or 30 mm to 45, 50, or 60 mm. When the central portion 186 is substantially unattached to the undergarment of the wearer and can move in response to the alternating movement of the legs thereby isolating the anterior region 12 and the central region 16 of the absorbent article 10 and allowing these regions to generally stay in place during movement of the wearer.

In various embodiments, the fasteners of the fastening system 180 may include any suitable attachment means, such as, adhesive, cohesive, hooks, snaps, clips, or the like, or combinations thereof. In various embodiments, the first fastener 182 may be a mechanical hook material attached to the garment facing surface 38 of the liquid impermeable layer 36 in the first side portion 142 and the second fastener 184 may be a mechanical hook material attached to the garment facing surface 38 of the liquid impermeable layer 36 in the second side portion 144. In various embodiments, the first fastener 182 may be an adhesive attached to the garment facing surface 38 of the liquid impermeable layer 36 in the first side portion 142 and the second fastener 184 may be an adhesive attached to the garment facing surface 38 of the liquid impermeable layer 36 in the second side portion 144.

In various embodiments, the first fastener 182 and/or the second fastener 184 may be continuous materials that extend from the anterior region 12 of the absorbent article 10, through the central region 16, and into the posterior region 14. In various embodiments, the first fastener 182 and/or the second fastener 184 may be continuous hook materials that extend from the anterior region 12, through the central region 16, and into the posterior region 14. In various embodiments, the first fastener 182 and/or the second fastener 184 may be continuous adhesive materials that extend from the anterior region 12, through the central region 16, and into the posterior region 14. In various embodiments, the first fastener 182 and/or the second fastener 184 may be separate and distinct materials positioned only in the posterior region 14.

In various embodiments, the first fastener 182 may be applied to the garment facing surface 38 of the liquid impermeable layer 36 in the first side portion 142 and at an angle relative to the longitudinal centerline 26. In various embodiments, the second fastener 184 may be applied to the garment facing surface 38 of the liquid impermeable layer 36 in the second side portion 144 and at an angle relative to the longitudinal centerline 26. In various embodiments, the first fastener 182 may be applied at an angle relative to the longitudinal centerline 26 to be generally parallel with the second flexure 120. In various embodiments, the second fastener 184 can be applied at an angle relative to the longitudinal centerline 26 to be generally parallel with the third flexure 130.

Wings:

In various embodiments, the absorbent article 10 can have a pair of wings 200 extending outwardly, in the transverse direction (Y), from the absorbent article 10. The wings 200 can drape over the edges of the wearer's undergarment so that the wings 200 are disposed between the edges of the wearer's undergarment and her thighs. The wings 200 can serve at least two purposes. First, the wings 200 can prevent soiling of the wearers undergarment by forming a barrier along the edges of the undergarment. Second, the wings 200 can be provided with fastener 190 of the fastening system 180, such as, for example, a garment attachment adhesive or a hook, to keep the absorbent article 10 securely and properly positioned in the undergarment. The wings 200 can wrap around the crotch region of the wearers undergarment to aid in securing the absorbent article 10 to the wearers undergarment when in use. Each wing 200 can fold under the crotch region of the wearer's undergarment and the fastener can either form a secure attachment to the opposite wing 200 or directly to the surface of the wearers undergarment. In various embodiments, the wings 200 can be an extension of materials forming the topsheet layer 30 and/or the liquid impermeable layer 36 and can be bonded together along the sealed peripheral region. Such wings 200 can be integrally formed with the main portion of the absorbent article 10. In various embodiments, the wings 200 can be constructed of materials similar to the topsheet layer 30, the liquid impermeable layer 36, or combinations of these materials. In various embodiments, the wings 200 can be separate elements bonded to the main body of the absorbent article 10. Examples of processes for manufacturing absorbent articles 10 and wings 200 include, but are not limited to, those described in U.S. Pat. No. 4,059,114 to Richards, U.S. Pat. No. 4,862,574 to Hassim, et al., U.S. Pat. No. 5,342,647 to Heindel, et al., U.S. Pat. No. 7,070,672 to Alcantara, et al., U.S. Publication No. 2004/0040650 to Venturino, et al., and international publication WO1997/040804 to Emenaker, et al., each of which are hereby incorporated by reference thereto in its entirety. It is to be understood that the wings 200 are optional and, in various embodiments, an absorbent article 10 can be configured without wings 200.

Tented Configuration:

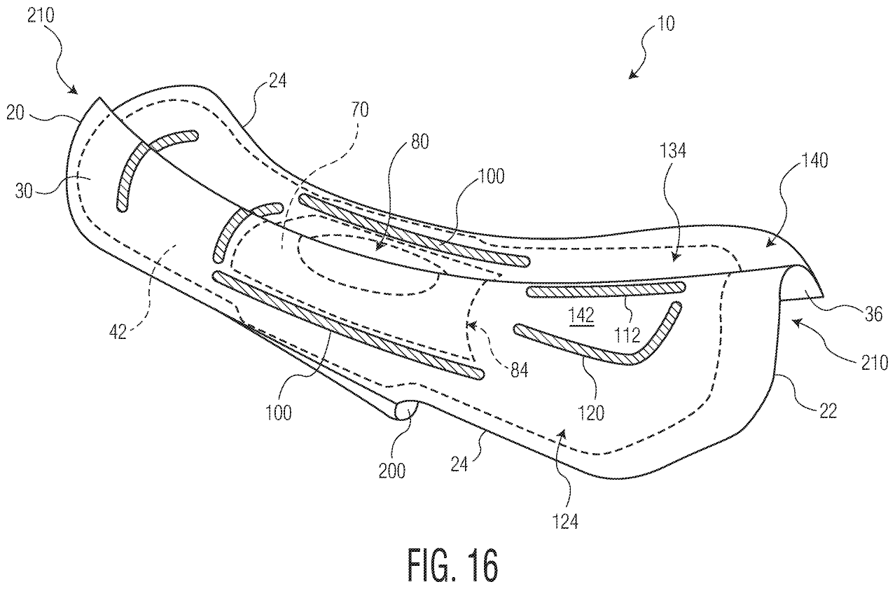

Referring to FIG. 16, the absorbent article 10 of FIG. 10 is illustrated in a tended configuration 210. The tented configuration 210 is formed through the entire longitudinal direction (X) length of the absorbent article 10 when the absorbent article 10 is subjected to lateral compression force. The tented configuration 210 in the anterior region 12 and central region 16 is formed relative to the presence of the slits 50 and crease 60 in the body conforming layer 38 of the absorbent assembly 40. The tented configuration 210 in the posterior region 14 is formed relative to the presence of the slits 50 and the crease 60 in the body conforming layer 38 as well as the first flexure 112, the second flexure 120, and the third flexure 130. The tented configuration 210 is adapted to conform to the body of the wearer for the purpose of intercepting body exudates moving along the skin of the wearer and/or preventing body exudates from following the body contours and escaping the absorbent article 10. In the posterior region 14, the tented configuration 210 is shaped by the rearward facing arch 84 of the fluid intake layer 70, the first flexure 112, the second flexure 120, and the third flexure 130. The tented configuration 210 is dynamic in the posterior region 14 due to the lack of attachment to the wearer's undergarment in the central portion 186 of the liquid impermeable layer 36. The first flexure 112 is believed to act as a lever to assist in lifting the tented configuration 210 to provide contact against the body of the wearer. The lifting force is generated by the reciprocating motion of the legs. This reciprocating motion alternately pumps the first side 142 of the dynamic region 140 and the second side 144 of the dynamic region towards the gluteal cleft. Generally the compressive force of the legs alternates when walking or running. In these situations, the compressive force alternates between the two sides of the absorbent article 10. The compressive force pushes alternately on the lateral stiffeners 100. The compressive force is transferred to the slits 50, the crease 60, and the dynamic region 140 in the posterior region 14 of the absorbent article 10. The absorbent material surround each of the slits 50 can shift in response to the compressive force and the apex of the crease 60 can lift upwards towards the topsheet layer 30 of the absorbent article 10 to form the tented configuration 210 in the anterior region 12 and central region 16 of the absorbent article 10. In the posterior region 14, the first side 142 of the dynamic region 140 acts as a plane that works in conjunction with a lateral stiffener 100 to push the first side 142 up into the tented configuration 210. Likewise, the second side 144 of the dynamic region 140 acts as a plane that works in conjunction with a lateral stiffener 100 to push the second side 144 up into the tented configuration 210. In various embodiments, such as, for example, wherein the absorbent article has a fastening system 180 such as illustrated in FIG. 14, the first side 142 and the second side 144 are free to move in response to the lateral forces because the central portion 186 of the liquid impermeable layer 36 is substantially free of fasteners in the fastening system 180. Because the first fastener 182 is positioned in the first side portion 124, the first side 142 is pulled back out when the compressive force is removed. Likewise, because the second fastener 184 is positioned in the second side portion 134, the second side 144 is pulled back out when the compressive force is removed. This configuration allows the absorbent article 10 to respond to the various compressive forces when the wearer is active.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any documents is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

What is claimed is:

1. An absorbent article comprising:
a. A topsheet layer;
b. A liquid impermeable layer;
c. An absorbent assembly positioned between the topsheet layer and the liquid impermeable layer and comprising a body conforming layer wherein the body conforming layer comprises a crease and a first plurality of slits.

2. The absorbent article of claim 1 wherein the first plurality of slits are arranged randomly in the body conforming layer.

3. The absorbent article of claim 1 wherein the first plurality of slits are arranged in a longitudinal direction of the absorbent article.

4. The absorbent article of claim 3 wherein the first plurality of slits are arranged in a first longitudinal direction column.

5. The absorbent article of claim 4 wherein the body conforming layer further comprises a second plurality of slits arranged in a longitudinal direction of the absorbent article.

6. The absorbent article of claim 5 wherein the second plurality of slits are arranged in a second longitudinal direction column.

7. The absorbent article of claim 6 wherein the first plurality of slits arranged in the first longitudinal direction column are offset in a transverse direction of the absorbent article from the second plurality of slits arranged in the second longitudinal direction column.

8. The absorbent article of claim 6 wherein the first plurality of slits arranged in the first longitudinal direction column at least partially overlap in a transverse direction of the absorbent article the second plurality of slits arranged in the second longitudinal direction column.

9. The absorbent article of claim 1 wherein the crease is centered on a longitudinal direction centerline of the absorbent article.

10. The absorbent article of claim 1 wherein the first plurality of slits overlap the crease.

11. The absorbent article of claim 1 wherein the absorbent assembly further comprises a second absorbent layer positioned between the topsheet layer and the body conforming layer.

12. The absorbent article of claim 1 wherein the absorbent assembly further comprises a second absorbent layer positioned between the body conforming layer and the liquid impermeable layer.

13. The absorbent article of claim 1 further comprising a fluid intake layer positioned between the topsheet layer and the absorbent assembly.

14. The absorbent article of claim 1 further comprising a fluid distribution layer positioned between the topsheet layer and the absorbent assembly.

15. The absorbent article of claim 1 further comprising a first flexure in a posterior region of the absorbent article extending in a direction parallel to a longitudinal centerline.

16. The absorbent article of claim 15 further comprising a second flexure in the posterior region of the absorbent article and spaced transverse outward in a first direction from the first flexure, the second flexure defining a first side portion of the absorbent article transversely outward in the first direction from the second flexure, and a third flexure in the posterior region of the absorbent article and spaced transversely outward in a second direction from the first flexure, the second direction being opposite the first direction, the third flexure defining a second side portion of the absorbent article transversely outward in the second direction from the third flexure, the second and third flexures defining a central portion of the absorbent article therebetween.
